(19) United States
(12) Patent Application Publication
(10) Pub. No.: US 2007/0166339 A1
(43) Pub. Date: Jul. 19, 2007

(54) Skin Whitening Methods and Compositions Based on Zeolite - Active Oxygen Donor Complexes

(75) Inventor: Shyam K. Gupta, Scottsdale, AZ (US)

Correspondence Address: SHYAM K. GUPTA;BIODERM RESEARCH, 5221 E. WINDROSE DRIVE, SCOTTSDALE, AZ, 85254, US

(73) Assignee: BIODERM RESEARCH, SCOTTSDALE, AZ (US)

(21) Appl. No.: 11/684,702

(22) Filed: Mar. 12, 2007

Related U.S. Application Data
(63) Continuation-in-part of application No. 10/710,011, filed on Jun. 11, 2004.
(63) Continuation-in-part of application No. 11/307,824, filed on Feb. 24, 2006.
Application No. 11/307,824 is a continuation-in-part of application No. 10/418,495, filed on Apr. 18, 2003, now abandoned.

(51) Int. Cl. A61K 8/02

(52) U.S. Cl. 424/401

(57) ABSTRACT

The present invention discloses certain complexes of anionic zeolites with active oxygen donor agents, such as organic or inorganic peroxides, and methods and compositions based on said complexes for skin whitening.

This is a continuation-in-part of U.S. patent application Ser. No. 10/710,011; filed on Jun. 11, 2004. This is also a continuation-in-part of U.S. patent application Ser. No. 11/307,824; filed on Feb. 24, 2006, which is a continuation-in-part of U.S. patent application Ser. No. 10/418,495; filed on Apr. 18, 2003, now abandoned.

U.S. patent application Ser. No. 11/307,824; filed on Feb. 24, 2006 by the present inventor disclosed the application of certain cage complexes of zeolites for topical controlled delivery of organic cosmetic and pharmaceutical active agents. Topical treatments that include skin aging, anti-wrinkle, antioxidants, skin whitening, acne, rosacea, sun screens, UV blocks, anesthetics, skin soothers, anti-irritants, anti-inflammatory agents, vitamins, hormones, and such that require a controlled release of such active agents were made practical by said cage complexes. Although controlled release of certain skin whitening agents was disclosed, the application of said zeolite cage complexes themselves for skin whitening was not disclosed.

The present invention discloses complexes of anionic zeolites with certain active oxygen donor agents such as organic and inorganic peroxides, the incorporation of said complexes in certain topical compositions, and the application of said complexes and said compositions for skin whitening, and skin discoloration and age spot reduction.

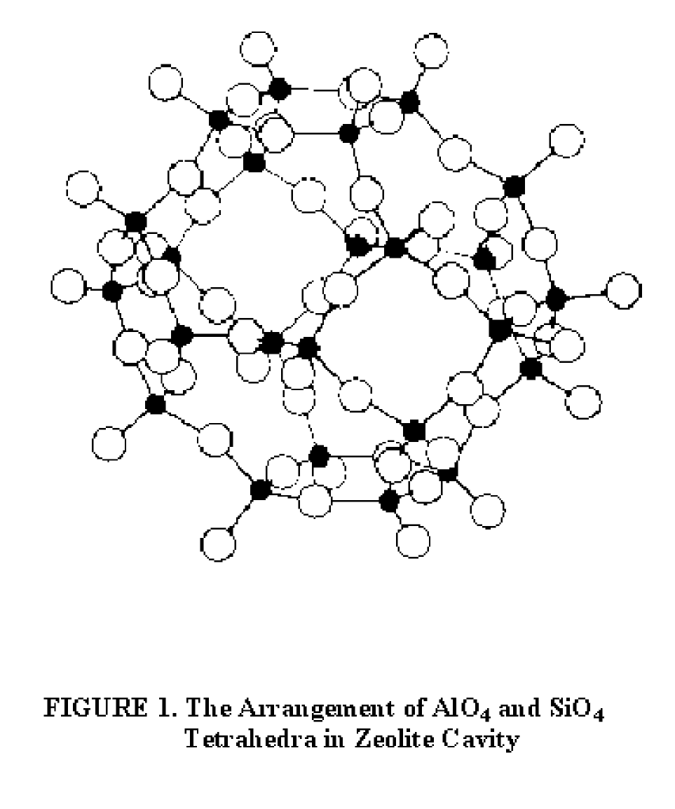

Zeolites are a group of crystalline aluminosilicates that have a porous, cage-like structure with a cavity. A zeolite may be defined as an aluminosilicate with a framework structure enclosing cavities occupied by large ions and water molecules, both of which have considerable freedom of movement, permitting ion-exchange and reversible dehydration. The framework consists of an open arrangement of corner-sharing tetrahedral where SiO4 are partially replaced by AlO4 tetrahedra, which requires sufficient cations to achieve electro neutrality [FIG. 1].

[FIG. 1].

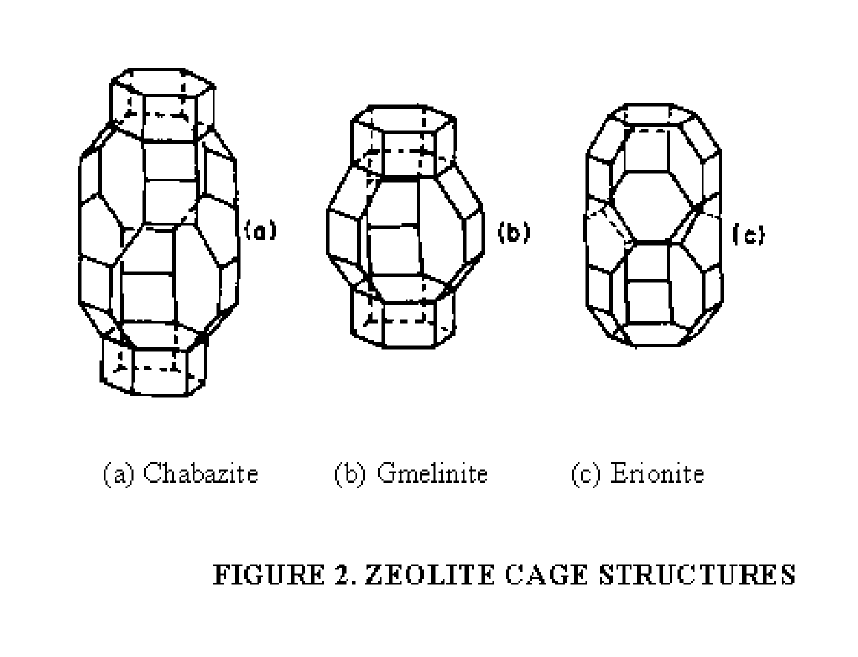

There are some 50 natural and over 150 synthetic zeolites, the latter all made by hydrothermal synthesis. The main uses are as molecular sieves, catalysts, and catalyst support for platinum group metals. Zeolite cavities are usually occupied by water. Some typical cavities occurring in Zeolite cages are shown in FIG. 2.

[FIG. 2].

Dehydration of synthetic zeolites leaves cubic micro crystals in which AlO4 and SiO4 tetrahedra are linked together to form a ring of eight O atoms on each face of the unit cube and an irregular ring of six O atoms across each corner. In the center of the unit cell is a large cavity about 11.4 Angstroms in diameter, which is connected to six identical cavities in adjacent unit cells by the eight-membered rings, which have inner diameter of about 4.2 Angstroms. In addition, the large cavity is connected to eight smaller cavities, about 6.6 Angstroms in diameter, by the six-membered rings, which provide openings of about 2.0 Angstrom in diameter. In the hydrated form all the cavities contain water molecules. In the anhydrous state the same cavities may be occupied by other molecules brought into contact with the zeolite, provided such molecules are able to pass through the apertures connecting cavities. Molecules within the cavities then tend to be held there by attractive forces of electrostatic and van der Waals types. Thus the zeolites will be able to absorb and strongly retain molecules just small enough to enter the cavities. It will not absorb at all those too big to enter. It will absorb weakly very small molecules that can enter or leave easily, except water molecules, which bind strongly.

The preparation and properties of anionic zeolites are described in detail in U.S. Pat. No. 2,882,243, among other sources. Generally, the preparation involves combining aqueous solutions that are sources of silica, alumina and sodium to produce a gel that crystallizes upon hydrothermal treatment. Conventional washing and drying steps provide hydrated Zeolite Na. The hydrated Zeolite Na must be modified with the substitution of potassium for part of the sodium to form Zeolite K prior to activation. The potassium modification is carried out by ion exchange in aqueous solution using nearly any appropriate potassium salt such as potassium chloride, potassium nitrate, potassium sulfate, and the like. The exchange can be carried out in any convenient manner that allows control of the amount of potassium exchanged for sodium, or for sodium with other metals. Heating of the hydrated Zeolite K to a temperature above about 300 C. provides a zeolite that has a strong heat of hydration.

The present invention discloses a method for the preparation of complexes of zeolites with certain active oxygen donor agents. The present invention further discloses the application of said complexes for the preparation of topical compositions suitable for skin whitening. The present invention additionally discloses a method for skin whitening via topical application of either the said zeolite complexes or said compositions based on said zeolite complexes. The skin whitening effect is more uniform, and does not cause skin irritation.

The complexes of active oxygen donor agents with anionic zeolites of the present invention also provide an unexpected and surprising topical release of active oxygen upon contact of said complexes with water molecules. The water molecules may be applied externally, or they may be provided by the natural perspiration of skin.

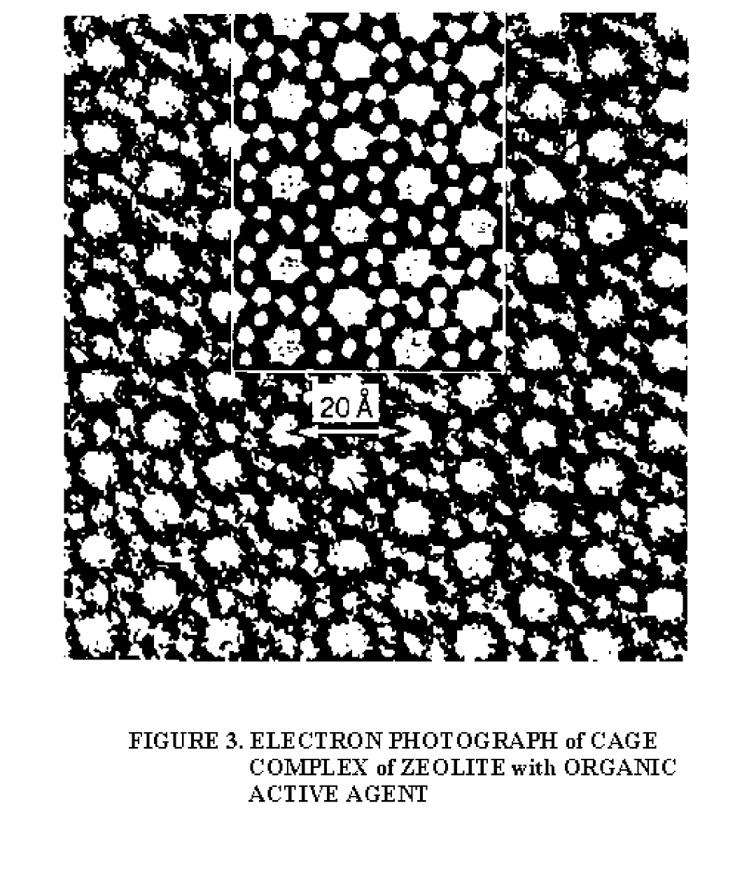

The said zeolite and active oxygen donor agent complexes can be prepared by mixing of an anionic zeolite with an active oxygen donor agent. The active oxygen donor agent can be either organic or inorganic in its composition. The organic active agent enters the zeolite cavity and forms a complex; the electron microscope photograph of one of such anionic Zeolite is shown in FIG. 3.

[FIG. 3].

The active oxygen donor agent can be either organic or inorganic in its composition.

The examples of inorganic active oxygen donors include hydrogen peroxide, sodium peroxide, calcium peroxide, sodium percarbonate, sodium persulfate, ammonium persulfate, barium peroxide, magnesium peroxide, lithium peroxide, zinc peroxide, and such.

The examples of organic active oxygen donors include urea peroxide, urea hydrogen peroxide, dibenzoyl peroxide, meta-chloroperbenzoic acid, peracetic acid, sodium peracetate, sodium meta-chloroperbenzoate, 2-butanone peroxide, di-tert-amyl peroxide, di-tert-butyl peroxide, dicumyl peroxide, dilauroyl peroxide, 2,4-Pentanedione peroxide, and such.

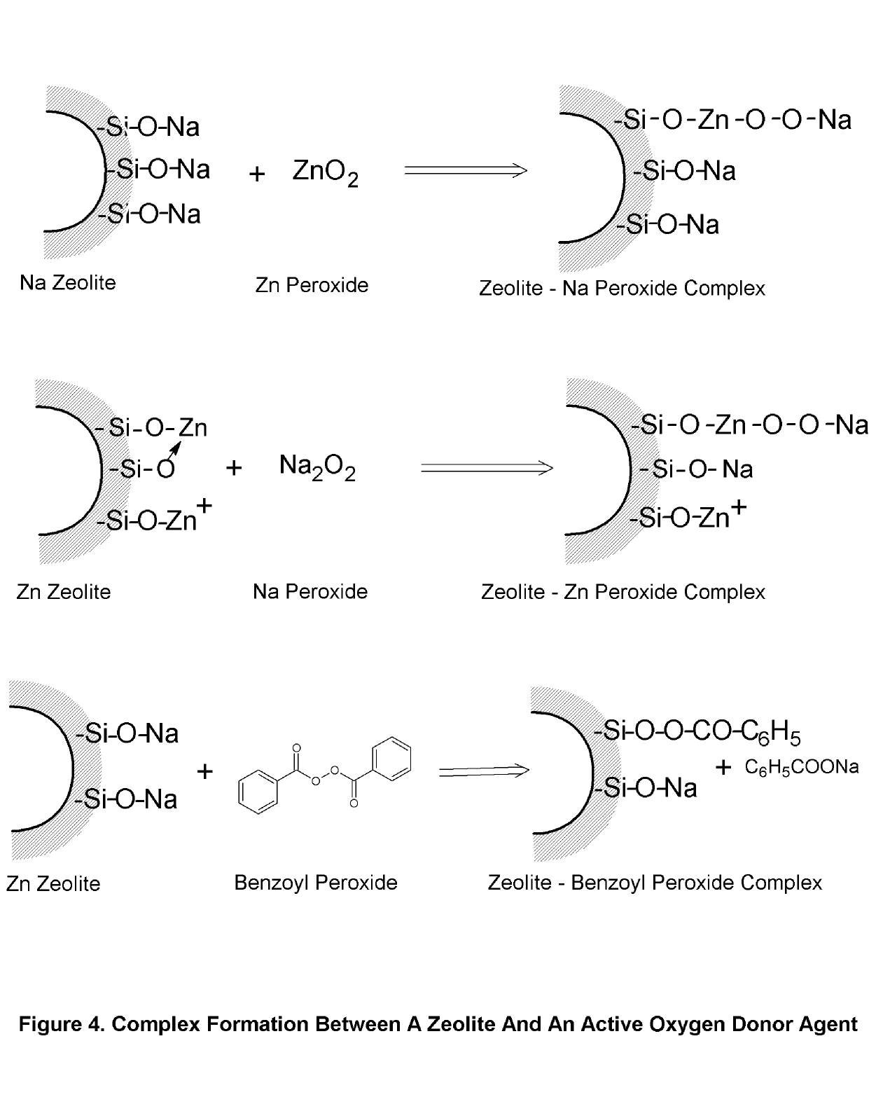

The proper selection of cation in the zeolite moiety and cation in the active oxygen donor moiety for the formation of stable zeolite-peroxide complexes is critically important. For example, the combination of sodium form of zeolite with hydrogen peroxide as the active oxygen donor agent leads to unstable zeolite-peroxide complex. However, the use of a monovalent zeolite, such as Na or K zeolite, and a divalent metal cation active oxygen donor, such as zinc peroxide, leads to the formation of corresponding zeolite-peroxide complex that is stable. Similarly, the use of a divalent metal cation zeolite, such as zinc zeolite, and a monovalent metal cation active oxygen donor, such as sodium peroxide, leads to the formation of corresponding zeolite-peroxide complex that is also stable. The complexation of sodium zeolite with benzoyl peroxide similarly results in a stable complex. These examples are further illustrated in [FIG. 4].

[FIG. 4].

Although the active oxygen donors themselves can be expected to bleach skin, those versed in the art also know that such a bleaching action can cause simultaneous skin irritation, which often can be painful. Moreover, such bleaching action cannot be controlled, as it can be almost instantaneous, depending on the method of its application. The skin whitening effect of zeolite complexes of active oxygen donor agents of the present invention is thus both surprising and unexpected, since said skin whitening effect is more controlled and uniform, as the active oxygen is released in a long-acting manner, i.e. over a prolonged period in a slow-release mode, and causes no skin irritation. Also, it does not require the preparation of two separate compositions, which is typical of prior art compositions that contain peroxides for topical application, for example, Lee et al. (WO 2007013735).

Zeolites can be made with both specific pore structures and bound cations such as Na, K, Mg, Ca, and Zn, that have found applications in various self-warming cosmetic compositions in the prior art. U.S. Pat. No. 3,250,680 (Menkart et al.) discloses applications of Zeolites for the preparation of self-heating toothpaste and other such compositions. Menkart utilizes only the heat releasing property of zeolites.

The combination of zeolites with certain active oxygen donor agents, such as hydrogen peroxide, has been disclosed in the prior art for several applications unrelated to the present invention.

Catinat et al. (U.S. Pat. Nos. 6,590,112; 6,380,407), for example, disclose a continuous process for manufacturing an oxirane, according to which an olefin is reacted at a temperature above 35.degree. C. and for a period of more than 48 hours, with hydrogen peroxide in the presence of a zeolite-based catalyst and in the presence of a metal salt, in which the catalyst undergoes no regeneration treatment and in which the rate of deactivation of the catalyst, expressed as being a percentage of the conversion of the peroxide compound lost per gram of oxirane produced per gram of zeolite determined after establishing the reaction conditions, i.e. after the consumption of 2.5 mol of peroxide function per gram of zeolite, is less than or equal to 0.15 percent. Unexpectedly, the combination of zeolite and hydrogen peroxide did not cause skin whitening, when tested according to the present invention.

Lee et al. (WO 2007013735) disclose a toothpaste composition having tooth whitening effect, and more specifically, to a toothpaste composition which contains hydrogen peroxide, as peroxide releasing oxygen free radicals for the whitening effect, and silica with reduced contents of metal ions, to overcome the difficulty in long-term storage due to the release of oxygen free radicals resulting from degradation of peroxide by metal ions released from other components in the composition, and unsatisfactory tooth whitening effect. While toothpaste whitening causes the whitening of teeth by bleaching organic food particles and organic food stains, it is not known for Lee et al. invention to also cause skin whitening, as latter is the effect of decolorization of topical melanin.

Jung et al. (KR 200400807544) disclose a cosmetic composition comprising a first paste containing peroxide and a second paste containing chloride is provided. The composition whitens, disinfects and massages the skin without causing skin damage as well as decolorizes hair. The cosmetic composition comprises: 3 to 12 percent by weight of a first paste containing peroxide; and 5 to 20 percent by weight of a second paste containing chloride. The peroxide is one or more selected from the group consisting of hydrogen peroxide, barium peroxide, sodium perborate, calcium peroxide and urea peroxide. When using the first paste and the second paste together in a double wall structured vessel with a membrane or a special vessel, the peroxide and the chloride react together and generate nascent oxygen. Jung et al. invention thus relates to a combination of two active agents, a peroxide agent and a chloride agent. It also is inconvenient, as two separate compositions are required for whitening effect.

Benzaminson et al. (WO 2006098680) disclose the use of a hydrophobic zeolite, that contains an active component, especially a disinfection element, as ethanol, iodine, phenol, cresol or hydrogen peroxide, in a composition for non-medical treatment of the skin, for example as a deodorant. The invention also describes the use of a hydrophobic zeolite, that contains an active component, especially a disinfection element, as hydrogen peroxide, for manufacturing of a pharmaceutical preparation for treatment of skin related conditions and diseases, as skin infections. The hydrophobic zeolite is especially selected from that group that comprises silicalite, or hydrophobic ZMS-5, hydrophobic mordenite and hydrophobic zeolite Y. Banzaminson et al. do not disclose any skin whitening benefits of said compositions.

U.S. Pat. No. 5,476,660 (Somasundaran et al.) discloses certain compositions of chemically modified zeolites in which zeolite surface has been modified to a positively charged state (cationic) or a zwitterionic state. These chemically modified zeolites have a filamentous structure with outwardly protruding positively charged organocarbonyl groups and also outwardly protruding negatively charged organocarbonyl groups. These chemically modified zeolites are useful for the deposition of active agents, more specifically, anionic active agents. Somasundaran et al. do not disclose the combination of said zeolites with any active oxygen donor agents for skin whitening.

U.S. Pat. No. 6,752,998 (Verdrel-Lahaxe et al.) discloses an exothermic composition, which includes at least one zeolite; at least one surfactant; at least one magnesium or calcium halide; and a physiologically acceptable anhydrous medium. Verdrel-Lahaxe et al. utilize only the heat-releasing or rubefacient properties of zeolites and do not disclose any controlled topical release of active oxygen donor agents.

U.S. Pat. No. 4,626,550 (Hertzenberg) discloses certain personal care products such as lotions and creams that are prepared using potassium exchanged Zeolite A that is much less anionic in nature. These compositions are useful only for the release of heat, and the inclusion of active agents such as bodying agents, topical pain relievers, antiperspirants and others must be largely anhydrous and should not enter the structures of the zeolite to release heat (col. 3, line 50-57). Hertzenberg does not disclose any controlled topical release of such active oxygen donor agents.

U.S. Pat. No. 4,379,143 (Sherry et al.) discloses activated or partially activated zeolites that can be included in analgesic balms or ointments as improved replacements for rubefacients. Upon hydration, the zeolite becomes warm, thereby helping to relieve pains associated with various musculoskeletal problems. Varying the character of the liquid vehicle can control generation and maintenance of the heat of hydration of anhydrous zeolite. If a very quick release of heat is desired, a hydrophilic vehicle is used; if a slow, sustained heat release is desired, a hydrophobic vehicle is required. Intermediate and controlled performance can be introduced by altering the hydrophobic vehicle to provide some hydrophilic characteristics. Sherry et al. thus utilize only the heat-releasing or rubefacient properties of zeolites and do not disclose any controlled topical release of active oxygen donor agents.

U.S. Pat. No. 6,274,128 (Bergman et al.) discloses an essentially anhydrous hair conditioning composition that comprises zeolites of specific pore size larger than the critical diameter of a water molecule and both the carrier molecules and the hair conditioner molecules that have molecular diameters larger than the largest average pore size of the micro porous materials. Bergman et al. utilize only the heat-releasing or rubefacient properties of zeolites and do not disclose any controlled topical release of active oxygen donor agents.

U.S. Pat. No. 6,309,655 (Minnix) discloses a cosmetic composition comprising a self-heating component, self-indicating disintegrating granules comprised of water-insoluble polymer and a colorant, which gives users indications of the length of time the composition has been applied and the degree of mixing when in use. This application is thus aimed at self-heating properties of zeolites, and their length of heating effect. Minnix utilizes only the heat-releasing or rubefacient properties of zeolites and do not disclose any topical release of active oxygen donor agents.

U.S. Application 20010016201 (Janchitraponvej) discloses a yet another self-heating application of an anhydrous rinse-out hair care composition utilizing zeolites.

Self-warming compositions have also been made with various anhydrous alkali metal salts (Giani et al., U.S. Pat. No. 5,747,004). In self-warming formulations based on Zeolites, the pore size specification is typically very small, from 3 to 10 angstroms in diameter, as is the ratio between sodium and potassium cations bound to silicate anions of such zeolites. These formulations release heat upon contact with water. Water penetrates the pores of such Zeolites and hydrates the interior silicate atoms of Zeolite agglomerates. Such interaction of zeolite with water releases the heat of hydration. Most cosmetic lotion, cream, shampoo, and conditioner products also contain hydrophilic and lipophilic ingredients for skin and hair care benefits. Some of such ingredients tend to clog the pores of Zeolites, causing a reduction in the heat-release properties of such formulations. The examples of such fatty materials that can inhibit the heat release properties of zeolites include most surfactants used in shampoo and body wash applications; quaternary ammonium compounds used for hair conditioning applications; fatty esters used as emollients in skin lotion and cream applications, and other similar examples. While such clogging of zeolite pores by above mentioned ingredients, some of which are highly desirable active agents, was considered a problem, and those problems were solved in the prior art by the use of small pore size zeolites that permit the entrance of water molecules inside their cavity but not other larger size molecules, for example U.S. Pat. No. 6,274,128.

U.S. patent application Ser. No. 20050133049 (Fournier et al.) discloses filters, smoking articles, and methods for selectively removing one or more selected constituents from mainstream smoke. The filters comprise zeolite BETA. Fournier et al. did discover that certain organic agents can bind with zeolite, but they failed to utilize this knowledge in the development of methods for topical delivery of any active oxygen donor bound agents.

U.S. patent application Ser. No. 20050058597 (Corbin et al.) discloses a process to synthesize nano-size Zeolite A from an amorphous gel precursor, which can be synthesized via reaction of NaAlO.sub.2, NaOH, and tetraethoxysilane (TEOS). Zeolite A with particle sizes of about 150 nm was made by transformation of the amorphous precursor in [(CH.sub.3).sub.4NOH] solution with Zeolite-A seeding. The nano-sized Zeolite A has been used for detergents. Corbin et al. did not disclose the utility of such nano-sized zeolites in controlled topical delivery of any active oxygen donor agents.

It is worthy of note that although zeolites with many different cations, such as titanium, zinc, manganese, iron, and copper have been disclosed, any applications of such metal zeolites in topical delivery of active oxygen donor agents have not been disclosed. This lack of knowledge is of special importance, since zeolites with enhanced ion-exchange capacity are well known (U.S. Patent Application 20010053741, Mikko et al.; U.S. Pat. No. 5,935,891; Prior).

U.S. Pat. No. 6,503,740 (Alther et al.) discloses zeolites treated with an organic modification compound such as quaternary amines, pyridinium compounds, and phosphonium amines that are useful for water treatment applications.

U.S. Pat. No. 6,365,130 (Barry et al.) discloses zeolites exchanged with antimicrobial metals for a chewing gum application, or a laundry application (U.S. Pat. No. 6,454,813; Chan). Modified zeolites have been used for topical cancer therapy (U.S. Pat. No. 6,288,045; Kaufman).

Zeolites have a very large surface area that is ionic in its nature. This surface area covers both the outside of zeolite and the inside of zeolite's porous cavity. The size of the pores of this cavity determines the size of any molecules that can enter zeolite's internal cavity. Almost all prior art disclosures have focussed on the cavity of zeolite. Since molecules larger in size than zeolite's cavity cannot enter zeolite's internal surface area, the delivery of such molecules from zeolite has not been disclosed in the prior art. The present invention circumvents this difficulty, and it is now possible to provide a controlled topical release of active oxygen donor agents that may be larger in size than the cavity of zeolite. This is because of the new complex formation method disclosed in the present invention that allows the formation of a complex of an anionic zeolite with an active oxygen donor agent that may be larger in size than zeolite pore size; said cage complex now being formed with the outer anionic surface of said zeolite when said organic active agent is in contact with said surface of said zeolite. As electron photographs have shown, some parts of said larger size active oxygen donor agent do enter the cavity of zeolite to form a cage complex, while other parts of said active oxygen donor agent remain attached to the outer surface of zeolite. It is like an octopus, which can enter its longer arms into the cavity of a submerged rock to extract a prey, while the main part of octopus remains on the outside of that rock. This property of zeolite-active oxygen donor agent cage complex was not known to the prior art, as it became known to the present inventor mostly due to the availability of the electron photograph shown in FIG. 3.

The skin whitening agents are well known in the prior art, for example Gupta (U.S. patent application Ser. No. 10/862,037; filed on Jun. 5, 2004). Hydroquinone a very commonly utilized skin-whitening agent is a reducing agent. Kojic acid and arbutin are both classified as tyrosinase inhibitors. The compositions of the present invention are the first known examples of skin-whitening agents that are based on oxidation via active oxygen donation by a stable active oxygen donor complex.

The efficacy of color reduction of melanin by active oxygen oxidation method of the present invention is established by an in-vitro test. Soluble melanin, which is commercially available, is reacted with zeolite-active oxygen donor agent complex in water medium, and the loss of melanin color is plotted. This data can be compared to a test similarly conducted with prior art skin whitening agents such as hydroquinone.

The compositions of the present invention can be formulated in various cosmetic and pharmaceutical consumer products utilizing a variety of delivery systems and carrier bases. Such consumer product forms include the group consisting of shampoos, aftershaves, sunscreens, body and hand lotions, skin creams, liquid soaps, bar soaps, bath oil bars, shaving creams, conditioners, permanent waves, hair relaxers, hair bleaches, hair detangling lotion, styling gel, styling glazes, spray foams, styling creams, styling waxes, styling lotions, mousses, spray gels, pomades, shower gels, bubble baths, hair coloring preparations, conditioners, hair lighteners, coloring and non-coloring hair rinses, hair grooming aids, hair tonics, spritzes, styling waxes, band-aids, and balms.

In another preferred aspect, the delivery system or a carrier base are selected in the form of a lotion, cream, gel, spray, thin liquid, body splash, powder, compressed powder, tooth paste, tooth powder, mouth spray, paste dentifrice, clear gel dentifrice, mask, serum, solid cosmetic stick, lip balm, shampoo, liquid soap, bar soap, bath oil, paste, salve, collodion, impregnated patch, impregnated strip, skin surface implant, impregnated or coated diaper, and similar delivery or packaging form.

In another preferred aspect, the delivery system can be human body or hair decolorizing solution, decolorizing powder, decolorizing gel, decolorizing spray, decolorizing stick, decolorizing roll-on, decolorizing paste, decolorizing cream, decolorizing lotion, decolorizing aerosol, and other commonly marketed human body and hair decolorizing compositions, household decolorizing solution, decolorizing powder, decolorizing gel, decolorizing spray, carpet decolorizer, room decolorizer, and other commonly marketed household decolorizing compositions, animals and pets decolorizing solution, decolorizing powder, decolorizing gel, decolorizing spray, animals and pets carpet decolorizer, animals and pets room decolorizer, and other commonly marketed animal and pet decolorizing compositions.

In another preferred aspect, the delivery system can be traditional water and oil emulsions, suspensions, colloids, microemulsions, clear solutions, suspensions of nanoparticles, emulsions of nanoparticles, or anhydrous compositions.

Additional cosmetically or pharmaceutically beneficial ingredients can also be included in the compositions of the present invention, which can be selected from, but not limited to skin cleansers, cationic, anionic surfactants, non-ionic surfactants, amphoteric surfactants, and zwitterionic surfactants, skin and hair conditioning agents, vitamins, hormones, minerals, plant extracts, anti-inflammatory agents, collagen and elastin synthesis boosters, UVA/UVB sunscreens, concentrates of plant extracts, emollients, moisturizers, skin protectants, humectants, silicones, skin soothing ingredients, antimicrobial agents, antifungal agents, treatment of skin infections and lesions, blood microcirculation improvement, skin redness reduction benefits, additional moisture absorbents, analgesics, skin penetration enhancers, solubilizers, moisturizers, emollients, anesthetics, colorants, perfumes, preservatives, seeds, broken seed nut shells, silica, clays, beads, luffa particles, polyethylene balls, mica, pH adjusters, processing aids, and combinations thereof.

In another preferred aspect, the cosmetically acceptable composition further comprises one or more excipient selected from the group consisting of water, saccharides, surface active agents, humectants, petrolatum, mineral oil, fatty alcohols, fatty ester emollients, waxes and silicone-containing waxes, silicone oil, silicone fluid, silicone surfactants, volatile hydrocarbon oils, quaternary nitrogen compounds, amine functionalized silicones, conditioning polymers, rheology modifiers, antioxidants, sunscreen active agents, di-long chain amines from about C.sub.10 to C.sub.22, long chain fatty amines from about C.sub.10 to C.sub.22, fatty alcohols, ethoxylated fatty alcohols and phospholipids.

Representative saccharides include nonionic or cationic saccharides such as agarose, amylopectins, amyloses, arabinans, arabinogalactans, arabinoxylans, carageenans, gum arabic, carboxymethyl guar gum, carboxymethyl(hydroxypropyl) guar gum, hydroxyethyl guar gum, carboxymethyl cellulose, cationic guar gum, cellulose ethers including methyl cellulose, chondroitin, chitins, chitosan, chitosan pyrrolidone carboxylate, chitosan glycolate chitosan lactate, cocodimonium hydroxypropyl oxyethyl cellulose, colominic acid ([poly-N acetyl-neuraminic acid]), corn starch, curdlan, dermatin sulfate, dextrans, furcellarans, dextrans, cross-linked dextrans, dextrin, emulsan, ethyl hydroxyethyl cellulose, flaxseed saccharide (acidic), galactoglucomannans, galactomainans, glucomannans, glycogens, guar gum, hydroxy ethyl starch, hydroxypropyl methyl cellulose, hydroxy ethyl cellulose, hydroxy propyl cellulose, hydroxypropyl starch, hydroxypropylated guar gums, gellan gum, gellan, gum ghatti, gum karaya, gum tragancanth (tragacanthin), heparin, hyaluronic acid, inulin, keratin sulfate, konjac mannan, modified starches, laminarans, laurdimonium hydroxypropyl oxyethyl cellulose, okra gum, oxidized starch, pectic acids, pectin, polydextrose, polyquaternium-4, polyquaternium-10, polyquaternium-28, potato starch, protopectins, psyllium seed gum, pullulan, sodium hyaluronate, starch diethylaminoethyl ether, steardimonium hydroxyethyl cellulose, raffinose, rhamsan, tapioca starch, whelan, levan, scleroglucan, sodium alginate, stachylose, succinoglycan, wheat starch, xanthan gum, xylans, xyloglucans, and mixtures thereof. Microbial saccharides can be found in Kirk-Othmer Encyclopedia of Chemical Technology, Fourth Edition, Vol. 16, John Wiley and Sons, NY pp. 578-611 (1994), which is incorporated entirely by reference. Complex carbohydrates found in Kirk-Othmer Encyclopedia of Chemical Technology, Fourth Edition, Vol. 4, John Wiley and Sons, NY pp. 930-948, 1995 which is herein incorporated by reference.

The cosmetically acceptable composition of this invention may include surface-active agents. Surface-active agents include surfactants, which typically provide detersive functionality to a formulation or act simply as wetting agents. Surface-active agents can generally be categorized as anionic surface-active agents, cationic surface-active agents, nonionic surface-active agents, amphoteric surface-active agents and zwitterionic surface-active agents, and dispersion polymers.

Anionic surface-active agents useful herein include those disclosed in U.S. Pat. No. 5,573,709, incorporated herein by reference. Examples include alkyl and alkyl ether sulfates. Specific examples of alkyl ether sulfates which may be used In this invention are sodium and ammonium salts of lauryl sulfate, lauryl ether sulfate, coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 12 to about 16 carbon atoms and an average degree of ethoxylation of from about 1 to about 6 moles of ethylene oxide.

Another suitable class of anionic surface-active agents is the alkyl sulfuric acid salts. Important examples are the salts of an organic sulfuric acid reaction product of a hydrocarbon of the methane series, including iso-, neo-, and n-paraffins, having about 8 to about 24 carbon atoms, preferably about 12 to about 18 carbon atoms and a sulfonating agent, for example, sulfur trioxide or oleum, obtained according to known sulfonation methods, including bleaching and hydrolysis. Preferred are alkali metals and ammonium sulfated C.sub.12-38 n-paraffins.

Additional synthetic anionic surface-active agents include the olefin sulfonates, the beta-alkyloxy alkane sulfonates, and the reaction products of fatty acids esterified with isethionic acid and neutralized with sodium hydroxide, as well as succinamates. Specific examples of succinamates include disodium N-octadecyl sulfosuccinamate; tetrasodium N-(1,2-dicarboxyethyl)-N-octadecylsulfosuccinamate; diamyl ester of sodium sulfosuccinic acid; dihexyl ester of sodium sulfosuccinic acid; dioctyl esters of sodium sulfosuccinic acid.

Preferred anionic surface-active agents for use in the cosmetically acceptable composition of this invention include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, triethanolamine lauryl sulfate, triethanolamine lauryl sulfate, monoethanolamine cocoyl sulfate, monoethanolamine lauryl sulfate, sodium tridecyl benzene sulfonate, and sodium dodecylbenzene sulfonate.

Amphoteric surface-active agents which may be used in the cosmetically acceptable composition of this invention include derivatives of aliphatic secondary and tertiary amines, in which the aliphatic substituent contains from about 8 to 18 carbon atoms and an anionic water solubilizing group e.g., carboxy, sulfonate, sulfate, phosphate, or phosphonate. Representative examples include sodium 3-dodecyl-aminopropionate, sodium 3-dodecylaminopropane sulfonate, sodium lauryl sarcosinate, N-alkyltaurines such as the one prepared by reacting dodecylamine with sodium isethionate as described in U.S. Pat. No. 2,658,072, N-higher alkyl aspartic acids as described in U.S. Pat. No. 2,438,091, and the products sold under the trade name MIRANOL. as described in U.S. Pat. No. 2,528,378. Other sarcosinates and sarcosinate derivatives can be found in the CTFA Cosmetic Ingredient Handbook, Fifth Edition, 1988, page 42 incorporated herein by reference.

Quaternary ammonium compounds can also be used in the cosmetically acceptable composition of this invention as long as they are compatible in the compositions of the invention, wherein the structure is provided in the CTFA Cosmetic Ingredient Handbook, Fifth Edition, 1988, page 40. Cationic surface-active agents generally include, but are not limited to fatty quaternary ammonium compounds containing from about 8 to about 18 carbon atoms. The anion of the quaternary ammonium compound can be a common ion such as chloride, ethosulfate, methosulfate, acetate, bromide, lactate, nitrate, phosphate, or tosylate and mixtures thereof. The long chain alkyl groups can include additional or replaced carbon or hydrogen atoms or ether linkages. Other substitutions on the quaternary nitrogen can be hydrogen, hydrogen, benzyl or short chain alkyl or hydroxyalkyl groups such as methyl, ethyl, hydroxymethyl or hydroxyethyl, hydroxypropyl or combinations thereof.

Examples of quaternary ammonium compounds include but are not limited to: Behentrimonium chloride, Cocotrimonium chloride, Cethethyldimonium bromide, Dibehenyldimonium chloride, Dihydrogenated tallow benzylmonium chloride, disoyadimonium chloride, Ditallowdimonium chloride, Hydroxycetyl hydroxyethyl dimonium chloride, Hydroxyethyl Behenamidopropyl dimonium chloride, Hydroxyethyl Cetyldimonium chloride, Hydroxyethyl tallowdimonium chloride, myristalkonium chloride, PEG-2 Oleamonium chloride, PEG-5 Stearmonium chloride, PEG-15 cocoyl quaternium 4, PEG-2 stearalkonium 4, lauryltrimonium chloride; Quaternium-16; Quaternium-18, lauralkonium chloride, olealkmonium chloride, cetylpyridinium chloride, Polyquaternium-5, Polyquaternium-6, Polyquaternium-7, Polyquaternium-10, Polyquaternium-22, Polyquaternium-37, Polyquaternium-39, Polyquaternium-47, cetyl trimonium chloride, dilauryldimonium chloride, cetalkonium chloride, dicetyldimonium chloride, soyatrimonium chloride, stearyl octyl dimonium methosulfate, and mixtures thereof. Other quaternary ammonium compounds are listed in the CTFA Cosmetic Ingredient Handbook, First Edition, on pages 41-42, incorporated herein by reference.

The cosmetically acceptable compositions of the present invention may include long chain fatty amines from about C.sub.10 to C.sub.22 and their derivatives. Specific examples include dipalmitylamine, lauramidopropyldimethylamine, and stearamidopropyl dimethylamine. The cosmetically acceptable compositions of this invention may also include fatty alcohols (typically monohydric alcohols), ethoxylated fatty alcohols, and di-tail phospholipids, which can be used to stabilize emulsion or dispersion forms of the cosmetically acceptable compositions. They also provide a cosmetically acceptable viscosity. Selection of the fatty alcohol is not critical, although those alcohols characterized as having fatty chains of C.sub.10 to C.sub.32, preferably C.sub.14 to C.sub.22, which are substantially saturated alkanols will generally be employed. Examples include stearyl alcohol, cetyl alcohol, cetostearyl alcohol, myristyl alcohol, behenyl alcohol, arachidic alcohol, isostearyl alcohol, and isocetyl alcohol. Cetyl alcohol is preferred and may be used alone or in combination with other fatty alcohols, preferably with stearyl alcohol. When used the fatty alcohol is preferably included in the formulations of this invention at a concentration within the range from about 1 to about 8 weight percent, more preferably about 2 to about 6 weight percent. The fatty alcohols may also be ethoxylated. Specific examples include cetereth-20, steareth-20, steareth-21, and mixtures thereof.

Phospholipids such as phosphatidylserine and phosphatidylcholine, and mixtures thereof may also be included. When used, the fatty alcohol component is included in the formulations at a concentration of about 1 to about 10 weight percent, more preferably about 2 to about 7 weight percent.

Nonionic surface-active agents, which can be used in the cosmetically acceptable composition of the present invention, include those broadly defined as compounds produced by the condensation of alkylene oxide groups (hydrophilic in nature) with an organic hydrophobic compound, which may be aliphatic or alkyl aromatic in nature. Examples of preferred classes of nonionic surface-active agents are: the long chain alkanolamides; the polyethylene oxide condensates of alkyl phenols; the condensation product of aliphatic alcohols having from about 8 to about 18 carbon atoms, in either straight chain or branched chain configuration, with ethylene oxide; the long chain tertiary amine oxides; the long chain tertiary phosphine oxides; the long chain dialkyl sulfoxides containing one short chain alkyl or hydroxy alkyl radical of from about 1 to about 3 carbon atoms; and the alkyl polysaccharide (APS) surfactants such as the alkyl polyglycosides; the polyethylene glycol (PEG) glyceryl fatty esters.

Zwitterionic surface-active agents such as betaines can also be useful in the cosmetically acceptable composition of this invention. Examples of betaines useful herein include the high alkyl betaines, such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl)sulfopropyl betaine and the like; amidobetaines and amidosulfobetaines, wherein the RCONH(CH.sub.2).sub.3 radical is attached to the nitrogen atom of the betaine are also useful in this invention.

The anionic, cationic, nonionic, amphoteric or zwitterionic surface-active agents used in the cosmetically acceptable composition of this invention are typically used in an amount from about 0.1 to 50 percent by weight, preferably from about 0.5 to about 40 percent by weight, more preferably from about 1 to about 20 percent by weight.

The cosmetically acceptable composition of this invention may include humectants, which act as hygroscopic agents, increasing the amount of water absorbed, held and retained. Suitable humectants for the formulations of this invention include but are not limited to: acetamide MEA, ammonium lactate, chitosan and its derivatives, colloidal oatmeal, galactoarabinan, glucose glutamate, glerecyth-7, glygeryth-12, glycereth-26, glyceryth-31, glycerin, lactamide MEA, lactamide DEA, lactic acid, methyl gluceth-10, methyl gluceth-20, panthenol, propylene glycol, sorbitol, polyethylene glycol, 1,3-butanediol, 1,2,6-hexanetriol, hydrogenated starch hydrolysate, inositol, mannitol, PEG-5 pentaerythritol ether, polyglyceryl sorbitol, xylitol, sucrose, sodium hyaluronate, sodium PCA, and combinations thereof. Glycerin is a particularly preferred humectant. The humectant is present in the composition at concentrations of from about 0.5 to about 40 percent by weight, preferably from about 0.5 to about 20 percent by weight and more preferably from about 0.5 to about 12 percent by weight.

The cosmetically acceptable composition of this invention may include petrolatum or mineral oil components, which when selected will generally be USP or NF grade. The petrolatum may be white or yellow. The viscosity or consistency grade of petrolatum is not narrowly critical. Petrolatum can be partially replaced with mixtures of hydrocarbon materials, which can be formulated to resemble petrolatum in appearance and consistency. For example, mixtures of petrolatum or mineral oil with different waxes and the like may be combined. Preferred waxes include bayberry wax, candelilla wax, ceresin, jojoba butter, lanolin wax, montan wax, ozokerite, polyglyceryl-3-beeswax, polyglyceryl-6-pentastearate, microcrystalline wax, paraffin wax, isoparaffin, vaseline solid paraffin, squalene, oligomer olefins, beeswax, synthetic candelilla wax, synthetic carnauba, synthetic beeswax and the like may be blended together. Alkylmethyl siloxanes with varying degrees of substitution can be used to increase water retained by the skin. Siloxanes such as stearyl dimethicone, known as 2503 Wax, C30-45 alkyl methicone, known as AMS-C30 wax, and stearoxytrimethylsilane (and) stearyl alcohol, known as 580 Wax, each available from Dow Coming, Midland, Mich., USA. Additional alkyl and phenyl silicones may be employed to enhance moisturizing properties. Resins such as dimethicone (and) trimethylsiloxysilicate or Cyclomethicone (and) Trimethylsiloxysilicate fluid, may be utilized to enhance film formation of skin care products. When used, the petrolatum, wax or hydrocarbon or oil component is included in the formulations at a concentration of about 1 to about 20 weight percent, more preferably about 1 to about 12 weight percent. When used, the silicone resins can be included from about 0.1 to about 10.0 weight percent.

Emollients are defined as agents that help maintain the soft, smooth, and pliable appearance of skin. Emollients function by their ability to remain on the skin surface or in the stratum corneum. The cosmetically acceptable composition of this invention may include fatty ester emollients, which are listed in the International Cosmetic Ingredient Dictionary, Eighth Edition, 2000, p. 1768 to 1773. Specific examples of suitable fatty esters for use in the formulation of this invention include isopropyl myristate, isopropyl palmitate, caprylic/capric triglycerides, cetyl lactate, cetyl palmitate, hydrogenated castor oil, glyceryl esters, hydroxycetyl isostearate, hydroxy cetyl phosphate, isopropyl isostearate, isostearyl isostearate, diisopropyl sebacate, PPG-5-Ceteth-20, 2-ethylhexyl isononoate, 2-ethylhexyl stearate, C.sub.12 to C.sub.16 fatty alcohol lactate, isopropyl lanolate, 2-ethyl-hexyl salicylate, and mixtures thereof. The presently preferred fatty esters are isopropyl myristate, isopropyl palmitate, PPG-5-Ceteth-20, and caprylic/capric triglycerides. When used the fatty ester emollient is preferably included in the formulations of this invention at a concentration of about 1 to about 8 weight percent, more preferably about 2 to about 5 weight percent.

The compositions of this invention may also include silicone compounds. Preferably, the viscosity of the silicone component is from about 0.5 to about 12,500 cps. Examples of suitable materials are dimethylpolysiloxane, diethylpolysiloxane, dimethylpolysiloxane-diphenylpolysiloxane, cyclomethicone, trimethylpolysiloxane, diphenylpolysiloxane, and mixtures thereof. Dimethicone, a dimethylpolysiloxane end blocked with trimethyl units, is one preferred example. Dimethicone having a viscosity between 50 and 1,000 cps is particularly preferred. When used, the silicone oils are preferably included in the formulations of this invention at a concentration of 0.1 to 5 weight percent, more preferably 1 to 2 weight percent.

The cosmetically acceptable compositions of this invention may include volatile and non-volatile silicone oils or fluids. The silicone compounds can be either linear or cyclic polydimethylsiloxanes with a viscosity from about 0.5 to about 100 centistokes. The most preferred linear polydimethylsiloxane compounds have a range from about 0.5 to about 50 centistokes. One example of a linear, low molecular weight, volatile polydimethylsiloxane is octamethyltrisiloxane-200 fluid having a viscosity of about 1 centistoke. When used, the silicone oils are preferably included in the formulations of this invention at a concentration of 0.1 to 30 weight percent, more preferably 1 to 20 weight percent.

The cosmetically acceptable compositions of this invention may include volatile, cyclic, low molecular weight polydimethylsiloxanes (cyclomethicones). The preferred cyclic volatile siloxanes can be polydimethyl cyclosiloxanes having an average repeat unit of 4 to 6, and a viscosity from about 2.0 to about 7.0 centistokes, and mixtures thereof. Preferred cyclomethicones are available from Dow Corning, Midland, Mich., and from General Electric, Waterford, N.Y., USA. When used, the silicone oils are preferably included in the formulations of this invention at a concentration of 0.1 to 30 weight percent, more preferably 1 to 20 weight percent.

Silicone surfactants or emulsifiers with polyoxyethylene or polyoxypropylene side chains may also be used in compositions of the current invention. Preferred examples include dimethicone copolyols and 5225C Formulation Aids, available from Dow Coming, Midland, Mich., USA and Silicone SF-1528, available from General Electric, Waterford, N.Y., USA. The side chains may also include alkyl groups such as lauryl or cetyl. Preferred are lauryl methicone copolyol. 5200 Formulation Aid, and cetyl dimethicone copolyol, known as Abil EM-90, available from Goldschmidt Chemical Corporation, Hopewell, Va. Also preferred is lauryl dimethicone, known as Belsil LDM 3107 VP, available from Wacker-Chemie, Munchen, Germany. When used, the silicone surfactants are preferably included in the formulations of this invention at a concentration of 0.1 to 30 weight percent, more preferably 1 to 15 weight percent. Amine functional silicones and emulsions may be utilized in the present invention. Preferred examples include Dow Coming 8220, Dow Coming 939, Dow Coming 949, Dow Coming 2-8194, all available from Dow Coming, Midland, Mich., USA. Also preferred is Silicone SM 253 available from General Electric, Waterford, N.Y., USA. When used, the amine functional silicones are preferably included in the formulations of this invention at a concentration of 0.1 to 5 weight percent, more preferably 0.1 to 2.0 weight percent.

The cosmetically acceptable compositions of this invention may include volatile hydrocarbon oils. The volatile hydrocarbon comprises from about C.sub.6 to C.sub.22 atoms. A preferred volatile hydrocarbon is an aliphatic hydrocarbon having a chain length from about C.sub.6 to C.sub.16 carbon atoms. An example of such compound includes isohexadecane, under the trade name Permethyl 101A, available from Presperse, South Plainfield, N.J., USA. Another example of a preferred volatile hydrocarbon is C.sub.12 to C.sub.14 isoparaffin, under the trade name Isopar M, available from Exxon, Baytown, Tex., USA. When used, the volatile hydrocarbons are preferably included in the formulations of this invention at a concentration of 0.1 to 30 weight percent, more preferably 1 to 20 weight percent.

The cosmetically acceptable compositions of this invention may include cationic and ampholytic conditioning polymers. Examples of such include, but are not limited to those listed by the International Cosmetic Ingredient Dictionary published by the Cosmetic, Toiletry, and Fragrance Association (CTFA), 1101 17 Street, N.W., Suite 300, Washington, D.C. 20036. General examples include quaternary derivatives of cellulose ethers, quaternary derivatives of guar, homopolymers and copolymers of DADMAC, homopolymers and copolymers of MAPTAC and quaternary derivatives of starches. Specific examples, using the CTFA designation, include, but are not limited to Polyquaternium-10, Guar hydroxypropyltrimonium chloride, Starch hydroxypropyltrimonium chloride, Polyquaternium-4, Polyquaternium-5, Polyquaternium-6, Polyquaternium-7, Polyquaternium-14, Polyquaternium-15, Polyquaternium-22, Polyquaternium-24, Polyquaternium-28, Polyquaternium-32, Polyquaternium-33, Polyquaternium-36, Polyquaternium-37, Polyquaternium-39, Polyquaternium-45, Polyquaternium-47 and polymethacrylamidopropyltrimonium chloride, and mixtures thereof. When used, the conditioning polymers are preferably included in the cosmetically acceptable composition of this invention at a concentration of from 0.1 to 10 weight percent, preferably from 0.2 to 6 weight percent and most preferably from 0.2 to 5 weight percent.

The cosmetically acceptable composition of this invention may include one or more rheological modifiers. The rheological modifiers that can be used in this invention include high molecular weight crosslinked homopolymers of acrylic acid, and Acrylates/C10-30 Alkyl Acrylate Crosspolymer, such as the Carbopol and Pemulen series, both available from B. F. Goodrich, Akron, Ohio, USA; anionic acrylate polymers such as Salcare and cationic acrylate polymers such as Salcare SC96, available from Ciba Specialties, High Point, N.C., USA; Acrylamidopropyltrimonium chloride/acrylamide; Hydroxyethyl methacrylates polymers, Steareth-10 Allyl Ether/Acrylate Copolymer; Acrylates/Beheneth-25 Metacrylate Copolymer, known as Aculyn, available from International Specialties, Wayne, N.J., USA; Glyceryl Polymethacrylate, Acrylates/Steareth-20 Methacrylate Copolymer; bentonite; gums such as alginates, carageenans, gum acacia, gum arabic, gum ghatti, gum karaya, gum tragacanth, guar gum; guar hydroxypropyltrimonium chloride, xanthan gum or gellan gum; cellulose derivatives such as sodium carboxymethyl cellulose, hydroxyethyl cellulose, hydroxymethyl carboxyethyl cellulose, hydroxymethyl carboxypropyl cellulose, ethyl cellulose, sulfated cellulose, hydroxypropyl cellulose, methyl cellulose, hydroxypropylmethyl cellulose, microcrystalline cellulose; agar; pectin; gelatin; starch and its derivatives; chitosan and its derivatives such as hydroxyethyl chitosan; polyvinyl alcohol, PVM/MA copolymer, PVM/MA decadiene crosspolymer, poly(ethylene oxide) based thickeners, sodium carbomer, and mixtures thereof. When used, the rheology modifiers are preferably included in the cosmetically acceptable composition of this invention at a concentration of from 0.01 to 12 weight percent, preferably from 0.05 to 10 weight percent and most preferably from 0.1 to 6 weight percent.

The cosmetically acceptable composition of this invention may include one or more antioxidants, which include, but are not limited to ascorbic acid, BHT, BHA, erythorbic acid, bisulfite, thioglycolate, tocopherol, sodium metabisulfite, vitamin E acetate, and ascorbyl palmitate. The anti oxidants will be present at from 0.01 to 20 weight percent, preferably 0.5 to 10 weight percent and most preferably from 1.0 to 5.0 weight percent of the cosmetically acceptable composition.

The cosmetically acceptable composition of this invention may include one or more sunscreen active agents. Examples of sunscreen active agents include, but are not limited to octyl methoxycinnamate (ethylhexyl p-methoxycinnamate), octyl salicylate oxybenzone (benzophenone-3), benzophenone-4, menthyl anthranilate, dioxybenzone, aminobenzoic acid, amyl dimethyl PABA, diethanolamine p-methoxy cinnamate, ethyl 4-bis (hydroxypropyl)aminobenzoate, 2-ethylhexy1-2-cyano-3,3-diphenylacrylate, homomenthyl salicylate, glyceryl aminobenzoate, dihydroxyacetone, octyl dimethyl PABA, 2-phenylbenzimidazole-5-sulfonic acid, triethanolamine salicylate, zinc oxide, zinc zeolite, titanium zeolite, and titanium oxide, and mixtures thereof. The amount of sunscreen used in the cosmetically acceptable composition of this invention will vary depending on the specific UV absorption wavelength(s) of the specific sunscreen active(s) used and can be from 0.1 to 10 percent by weight, from 2 to 8 percent by weight.

The cosmetically acceptable composition of this invention may include one or more preservatives. Example of preservatives, which may be used include, but are not limited to 1,2-dibromo-2,4-dicyano butane (Methyldibromo Glutaronitrile, known as MERGUARD. Nalco Chemical Company, Naperville, Ill., USA), benzyl alcohol, imidazolidinyl urea, 1,3-bis (hydroxymethyl)-5,5-dimethyl-2,3-imidazolidinedione (e.g., DMDM Hydantoin, known as GLYDANT, Lonza, Fairlawn, N.J., USA.), methylchloroisothiazolinone and methylisothiazolinone (e.g., Kathon, Rohm & Haas Co., Philadelphia, Pa., USA), methyl paraben, propyl paraben, phenoxyethanol, and sodium benzoate, and mixtures thereof.

The cosmetically acceptable composition of this invention may include any other ingredient by normally used in cosmetics. Examples of such ingredients include, but are not limited to buffering agents, fragrance ingredients, chelating agents, color additives or dyestuffs which can serve to color the composition itself or keratin, sequestering agents, softeners, foam synergistic agents, foam stabilizers, sun filters and peptizing agents.

The surface of pigments, such titanium dioxide, zinc oxide, talc, calcium carbonate or kaolin, can be treated with the unsaturated quaternary ammonium compounds described herein and then used in the cosmetically acceptable composition of this invention. The treated pigments are then more effective as sunscreen actives and for use in color cosmetics such as make up and mascara.

The cosmetically acceptable composition of this invention can be presented in various forms. Examples of such forms include, but are not limited a solution, liquid, cream, emulsion, dispersion, gel, thickening lotion.

The cosmetically acceptable composition of this invention may contain water and also any cosmetically acceptable solvent. Examples of acceptable solvents include, but are not limited to monoalcohols, such as alkanols having 1 to 8 carbon atoms (like ethanol, isopropanol, benzyl alcohol and phenylethyl alcohol) polyalcohols, such as alkylene glycols (like glycerin, ethylene glycol and propylene glycol) and glycol ethers, such as mono-, di- and tri-ethylene glycol monoalkyl ethers, for example ethylene glycol monomethyl ether and diethylene glycol monomethyl ether, used singly or in a mixture. from 0.1 to 70 percent by weight, relative to the weight of the total composition.

The cosmetically acceptable composition of this invention can also be packaged as an aerosol, in which case it can be applied either in the form of an aerosol spray or in the form of an aerosol foam. As the propellant gas for these aerosols, it is possible to use, in particular, dimethyl ether, carbon dioxide, nitrogen, nitrous oxide, air and volatile hydrocarbons, such as butane, isobutane, and propane.

The cosmetically acceptable composition of this invention also can contain electrolytes, such as aluminum chlorohydrate, alkali metal salts, e.g., sodium, potassium or lithium salts, these salts preferably being halides, such as the chloride or bromide, and the sulfate, or salts with organic acids, such as the acetates or lactates, and also alkaline earth metal salts, preferably the carbonates, silicates, nitrates, acetates, gluconates, pantothenates and lactates of calcium, magnesium and strontium.

Compositions for treating skin include leave-on or rinse-off skin care products such as lotions, hand/body creams, shaving gels or shaving creams, body washes, sunscreens, liquid soaps, deodorants, antiperspirants, suntan lotions, after sun gels, bubble baths, hand or mechanical dishwashing compositions, and the like. In addition to the polymer, skin care compositions may include components conventionally used in skin care formulations. Such components include for example; (a) humectants, (b) petrolatum or mineral oil, (c) fatty alcohols, (d) fatty ester emollients, (e) silicone oils or fluids, and (f) preservatives. These components must in general be safe for application to the human skin and must be compatible with the other components of the formulation. Selection of these components is generally within the skill of the art. The skin care compositions may also contain other conventional additives employed in cosmetic skin care formulations. Such additives include aesthetic enhancers, fragrance oils, dyes and medicaments such as menthol and the like.

The skin care compositions of this invention may be prepared as oil-in-water, water-in-oil emulsions, triple emulsions, or dispersions.

Preferred oil-in-water emulsions are prepared by first forming an aqueous mixture of the water-soluble components, e.g. unsaturated quaternary ammonium compounds, humectants, water-soluble preservatives, followed by adding water-insoluble components. The water-insoluble components include the emulsifier, water-insoluble preservatives, petrolatum or mineral oil component, fatty alcohol component, fatty ester emollient, and silicone oil component. The input of mixing energy will be high and will be maintained for a time sufficient to form a water-in-oil emulsion having a smooth appearance (indicating the presence of relatively small micelles in the emulsion). Preferred dispersions are generally prepared by forming an aqueous mixture of the water-soluble components, followed by addition of thickener with suspension power for water-insoluble materials.

Compositions for treating hair include bath preparations such as bubble baths, soaps, and oils, shampoos, conditioners, hair bleaches, hair coloring preparations, temporary and permanent hair colors, color conditioners, hair lighteners, coloring and non-coloring hair rinses, hair tints, hair wave sets, permanent waves, curling, hair straighteners, hair grooming aids, hair tonics, hair dressings and oxidative products. The dispersion polymers may also be utilized in styling type leave-in products such as gels, mousses, spritzes, styling creams, styling waxes, pomades, balms, and the like, either alone or in combination with other polymers or structuring agents in order to provide control and hair manageability with a clean, natural, non-sticky feel.

Hair care compositions of this invention give slippery feel and that can be easily rinsed from the hair due to the presence of the dispersion polymer, volatile silicones, other polymers, surfactants or other compounds that may alter the deposition of materials upon the hair.

In the case of cleansing formulations such as a shampoo for washing the hair, or a liquid hand soap, or shower gel for washing the skin, the compositions contain anionic, cationic, nonionic, zwitterionic or amphoteric surface-active agents typically in an amount from about 3 to about 50 percent by weight, preferably from about 3 to about 20 percent, and their pH is general in the range from about 3 to about 10.

Preferred shampoos of this invention contain combinations of anionic surfactants with zwitterionic surfactants and/or amphoteric surfactants. Especially preferred shampoos contain from about 0 to about 16 percent active of alkyl sulfates, from 0 to about 50 weight percent of ethoxylated alkyl sulfates, and from 0 to about 50 weight percent of optional surface-active agents selected from the nonionic, amphoteric, and zwitterionic surface-active agents, with at least 5 weight percent of either alkyl sulfate, ethoxylated alkyl sulfate, or a mixture thereof, and a total surfactant level of from about 10 weight to about 25 percent.

The shampoo for washing hair also can contain other conditioning additives such as silicones and conditioning polymers typically used in shampoos. U.S. Pat. No. 5,573,709 provides a list of non-volatile silicone conditioning agents that can be used in shampoos. The conditioning polymers for use with the present invention are listed in the Cosmetic, Toiletries and Fragrance Associations (CTFA) dictionary. Specific examples include the Polyquaterniums (example Polyquaternium-1 to Polyquaternium-50), Guar Hydroxypropyl Trimonium Chloride, Starch Hydroxypropyl Trimonium Chloride and Polymethacrylamidopropyl Trimonium Chloride.

Other preferred embodiments consist of use in the form of a rinsing lotion to be applied mainly before or after shampooing. These lotions typically are aqueous or aqueous-alcoholic solutions, emulsions, thickened lotions or gels. If the compositions are presented in the form of an emulsion, they can be nonionic, anionic or cationic. The nonionic emulsions consist mainly of a mixture of oil and/or a fatty alcohol with a polyoxyethyleneated alcohol, such as polyoxyethyleneated stearyl or cetyl/stearyl alcohol, and cationic surface-active agents can be added to these compositions. The anionic emulsions are formed essentially from soap.

If the compositions are presented in the form of a thickened lotion or a gel, they contain thickeners in the presence or absence of a solvent. The thickeners which can be used are especially resins, Carbopol-type acrylic acid thickeners available from B.F. Goodrich; xanthan gums; sodium alginates; gum arabic; cellulose derivatives and poly-(ethylene oxide) based thickeners, and it is also possible to achieve thickening by means of a mixture of polyethylene glycol stearate or distearate or by means of a mixture of a phosphoric acid ester and an amide. The concentration of thickener is generally 0.05 to 15 percent by weight. If the compositions are presented in the form of a styling lotion, shaping lotion, or setting lotion, they generally comprise, in aqueous, alcoholic or aqueous-alcoholic solution, the ampholyte polymers defined above.

In the case of hair fixatives, the composition may also contain one or more additional hair fixative polymers. When present, the additional hair fixative polymers are present in a total amount of from about 0.25 to about 10 percent by weight. The additional hair fixative resin can be selected from the following group as long as it is compatible with a given dispersion polymer: acrylamide copolymer, acrylamide/sodium acrylate copolymer, acrylate/ammonium methacrylate copolymer, an acrylate copolymer, an acrylic/acrylate copolymer, adipic acid/dimethylaminohydroxypropyl diethylenetriamine copolymer, adipic acid/epoxypropyl diethylenetriamine copolymer, allyl stearate/VA copolymer, aminoethylacrylate phosphate/acrylate copolymer, an ammonium acrylate copolymer, an ammonium vinyl acetate/acrylate copolymer, an AMP acrylate/diacetoneacrylamide copolymer, an AMPD acrylate/diacetoneacrylamide copolymer, butyl ester of ethylene/maleic anhydride copolymer, butyl ester of PVM/MA copolymer, calcium/sodium PVM/MA copolymer, corn starch/acrylamide/sodium acrylate copolymer, diethylene glycolamine/epichlorohydrin/piperazine-copolymer, dodecanedioic acid/cetearyl alcohol/glycol copolymer, ethyl ester of PVM/MA copolymer, isopropyl ester of PVM/MA copolymer, karaya gum, a methacryloyl ethyl betaine/methacrylate copolymer, an octylacrylamide/acrylate/butylaminoethyl methacrylate copolymer, an octylacrylamide/acrylate copolymer, phthalic anhydride/glycerin/glycidyl decanoate copolymer, a phthalic/trimellitic/glycol copolymer, polyacrylamide, polyacrylamidomethylpropane sulfonic acid, polybutylene terephthalate, polyethylacrylate, polyethylene, polyquaternium-1, polyquaternium-2, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-39, polyquaternium-47, polyvinyl acetate, polyvinyl butyral, polyvinyl imidazolinium acetate, polyvinyl methyl ether, PVM/MA copolymer, PVP, PVP/dimethylaminoethylmethacrylate copolymer, PVP/eicosene copolymer, PVP/ethyl methacrylate/methacrylic acid copolymer, PVP/hexadecene copolymer, PVP/VA copolymer, PVP/vinyl acetate/itaconic acid copolymer, shellac, sodium acrylates copolymer, sodium acrylates/Acrylnitrogens copolymer, sodium acrylate/vinyl alcohol copolymer, sodium carrageenan, starch diethylaminoethyl ether, stearylvinyl ether/maleic anhydride copolymer, sucrose benzoate/sucrose acetate isobutyrate/butyl benzyl phthalate copolymer, sucrose benzoate/sucrose acetate isobutyrate/butyl benzyl phthalate/methyl methacrylate copolymer, sucrose benzoate/sucrose acetate isobutyrate copolymer, a vinyl acetate/crotonate copolymer, vinyl acetate/crotonic acid copolymer, vinyl acetate/crotonic acid/methacryloxybenzophenone-1 copolymer, vinyl acetate/crotonic acid/vinyl neodecanoate copolymer, and mixtures thereof. Synthetic polymers used for creating styling aids are described in “The History of Polymers in Haircare,” Cosmetics and Toiletries, 103 (1988), incorporated herein by reference. Other synthetic polymers that may be used with the present invention can be referenced in the CTFA Dictionary, Fifth Edition, 2000, incorporated herein by reference.

The cosmetic compositions of this invention may be formulated in a wide variety of form, for non-limited example, including a solution, a suspension, an emulsion, a paste, an ointment, a gel, a cream, a lotion, a powder, a soap, a surfactant-containing cleanser, an oil, a powder foundation, an emulsion foundation, a wax foundation and a spray. In detail, the cosmetic composition of the present invention can be provided in a form of skin softener (skin lotion), astringent lotion, nutrient emulsion (milk lotion), nutrient cream, message cream, essence, eye cream, cleansing cream, cleansing foam, cleansing water, facial pack, spray or powder.

The cosmetically acceptable carrier contained in the present cosmetic composition, may be varied depending on the type of the formulation. For example, the formulation of ointment, pastes, creams or gels may comprise animal and vegetable fats, waxes, paraffins, starch, tragacanth, cellulose derivatives, polyethylene glycols, silicones, bentonite, silica, talc, zinc oxide or mixtures of these ingredients.

In the formulation of powder or spray, it may comprise lactose, talc, silica, aluminum hydroxide, calcium silicate, polyamide powder and mixtures of these ingredients. Spray may additionally comprise the customary propellants, for example, chlorofluorohydrocarbons, propane, butane, diethyl ether, or dimethyl ether.

The formulation of solution and emulsion may comprise solvent, solubilizer and emulsifier, for example water, ethanol, isopropanol, ethyl carbonate, ethyl acetate, benzyl alcohol, benzyl benzoate, propylene glycol, 1,3-butyleneglycol, oils, in particular cottonseed oil, groundnut oil, maize germ oil, olive oil, castor oil and sesame seed oil, glycerol fatty esters, polyethylene glycol and fatty acid esters of sorbitan or mixtures of these ingredients.

The formulation of suspension may comprise liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminum metahydroxide, bentonite, agar and tragacanth or mixtures of these ingredients.

The formulation of cleansing compositions with surfactant may comprise aliphatic alcohol sulfate, aliphatic alcohol ether sulfate, sulfosucinnate monoester, isethionate, imidazolium derivatives, methyl taurate, sarcosinate, fatty acid amide ether sulfate, alkyl amido betaine, aliphatic alcohol, fatty acid glyceride, fatty acid diethanolamide, vegetable oil, lanoline derivatives, ethoxylated glycerol fatty acid ester or mixtures of these ingredients.

Additional antioxidant ingredients and compositions can be selected from, but not limited to, Ascorbic acid, Ascorbic acid derivatives, Glucosamine ascorbate, Arginine ascorbate, Lysine ascorbate, Glutathione ascorbate, Nicotinamide ascorbate, Niacin ascorbate, Allantoin ascorbate, Creatine ascorbate, Creatinine ascorbate, Chondroitin ascorbate, Chitosan ascorbate, DNA Ascorbate, Carnosine ascorbate, Vitamin E, various Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), α-Lipoic acid, Niacinamide lipoate, Glutathione, Andrographolide (Andrographis paniculata), Carnosine, Niacinamide, Potentilla erecta extract, Polyphenols, Grapeseed extract, Pycnogenol (Pine Bark extract), Pyridoxine, Magnolol, Honokiol, Paeonol, Resacetophenone, Quinacetophenone, arbutin, kojic acid, and combinations thereof.

The blood micro-circulation improvement ingredients and compositions can be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis Vinifera) leaves, apigenin, phytosan, luteolin, and combinations thereof.

The anti-inflammatory ingredients or compositions can be selected from, but not limited to, at least one antioxidant class of Cyclo-oxygenase (for example, COX-1 or COX-2) or Lipoxygenase (for example, LOX-5) enzyme inhibitors such as Ascorbic acid, Ascorbic acid derivatives, Vitamin E, Vitamin E derivatives, Tocotrienol, Rutin, Quercetin, Hesperedin (Citrus sinensis), Diosmin (Citrus sinensis), Mangiferin (Mangifera indica), Mangostin (Garcinia mangostana), Cyanidin (Vaccinium myrtillus), Astaxanthin (Haematococcus algae), Lutein (Tagetes patula), Lycopene (Lycopersicum esculentum), Resveratrol (Polygonum cuspidatum), Tetrahydrocurcumin (Curcuma longa), Rosmarinic acid (Rosmarinus officinalis), Hypericin (Hypericum perforatum), Ellagic acid (Punica granatum), Chlorogenic acid (Vaccinium vulgaris), Oleuropein (Olea europaea), alpha-Lipoic acid, Glutathione, Andrographolide, Grapeseed extract, Green Tea Extract, Polyphenols, Pycnogenol (Pine Bark extract), White Tea extract, Black Tea extract, (Andrographis paniculata), Carnosine, Niacinamide, and Emblica extract. Anti-inflammatory composition can additionally be selected from, but not limited to, Horse Chestnut Extract (Aesculus hippocastanum extract)), Esculin, Escin, Yohimbine, Capsicum Oleoresin, Capsaicin, Niacin, Niacin Esters, Methyl Nicotinate, Benzyl Nicotinate, Ruscogenins (Butchers Broom extract; Ruscus aculeatus extract), Diosgenin (Trigonella foenumgraecum, Fenugreek), Emblica extract (Phyllanthus emblica extract), Asiaticoside (Centella asiatica extract), Boswellia Extract (Boswellia serrata), Sericoside, Visnadine, Thiocolchicoside, Grapeseed Extract, Ginger Root Extract (Zingiber Officianalis), Piperine, Vitamin K, Melilot (Melilotus officinalis extract), Glycyrrhetinic acid, Ursolic acid, Sericoside (Terminalia sericea extract), Darutoside (Siegesbeckia orientalis extract), Amni visnaga extract, extract of Red Vine (Vitis-Vinifera) leaves, apigenin, phytosan, luteolin, and combinations thereof.

The zeolite-active oxygen donor complexes of the present invention can also be made in-situ by a combination of a suitable zeolite with a suitable oxygen donor agent. The in-situ combination of zinc zeolite with sodium peroxide, for example, leads to the formation of zinc peroxide-zeolite complex. The in-situ combination of Na/K zeolite with zinc peroxide also leads to the formation of zinc peroxide-zeolite complex. This is further illustrated in Examples 1 and 2 of the present invention. The removal of water from Examples 1 and 2 can be used for obtaining said complexes in dry state.

### EXAMPLES

The following examples illustrate presently preferred practice thereof. As illustrations they are not intended to limit the scope of the invention. All quantities are in weight percent.

### Example 1

### Preparation of Zinc Peroxide-Zeolite Complex from Na/K Zeolite

Ingredients. (1) Zeolite, Type 4A 20.0 (2) Zinc Peroxide 1.0 (3) Water 79.0. Procedure. Mix (2) and (3) to a clear solution. Add (1) and mix. The mixture contains 10.0 Mmol of Zinc Peroxide-zeolite complex (100% zeolite Na exchanged).

### Example 2

### Preparation of Zinc Peroxide-Zeolite Complex from Zinc Zeolite

Ingredients. (1) Zinc Zeolite 10.0 (2) Sodium Peroxide 3.0 (3) Water 87. Procedure. Mix (2) and (3) to a clear solution. Add (1) and mix. The mixture contains about 30 Mmol of Zinc Peroxide-zeolite complex.

### Example 3

### Preparation of Benzoyl Peroxide-Zeolite Complex

Ingredients. (1) Zeolite, Type 4A 40.0 (2) Benzoyl Peroxide 2.4 (3) PEG-6 57.6. Procedure. Mix (2) and (3) to a clear solution. Add (1) and mix. The mixture contains 10 Mmol of Benzoyl Peroxide-zeolite complex.

### Example 4

### Skin Whitening Serum

Ingredients. (1) Ethyl Lactate 20.0 (2) Polyalkyleneoxy Polyamide 0.5 (3) Zinc peroxide-Zeolite complex 9.0 (4) PEG-6 70.0 (5) Preservatives 0.5. Procedure. Make serum base by mixing (1), (2) and (4) at 60 to 70 C. Cool to 30 to 40 C and add (3) to main batch with mixing.

### Example 5

### Anti-Acne and Facial Oil Control Cream

Ingredients. (1) Deionized water 79.5 (2) Cetearyl alcohol (and) dicetyl phosphate (and) Ceteth-10 phosphate 5.0 (3) Cetyl alcohol 2.0 (4) Glyceryl stearate (and) PEG-100 stearate 4.0 (5) Ethyl Lactate 5.0 (6) Zinc Peroxide-Zeolite complex 4.0 (7) Preservatives 0.5. Procedure. Mix 1 to 5 and heat to 75-80° C. Adjust pH to 4.0 4.5. Cool to 35-40 C with mixing. Add 6 to 7 with mixing. An off-white cream is obtained.

### Example 6

### Skin Decolorizing and Age Spots Cream

Ingredients. (1) Water 53.9 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (60) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Water 5.0 (15) Benzoyl Peroxide-Zeolite complex 8.1 (16) Artemisinin 0.5 (17) Carnosine 0.1 (18) Cyclomethicone, Dimethicone Crosspolymer 2.0 (19) Polysorbate-20 2.0 (20) Ethyl Lactate 12.0. Procedure. Mix (1) to (13) and heat at 70 to 80 C till homogenous. Cool to 40 to 50 C. Premix and add all other ingredients to main batch and mix. Cool to room temperature. An off-white cream is obtained.

### Example 7

### Skin Whitening Cream

Ingredients. (1) Water 53.8 (2) Dicetyl Phosphate (and) Ceteth-10 Phosphate 5.0 (3) Glyceryl Stearate (and) PEG-100 Stearate 4.0 (4) Phenoxyethanol 0.7 (5) Chlorphenesin 0.3 (6) Titanium Dioxide 0.2 (7) Sodium Hydroxide 0.5 (8) Magnolol 0.2 (9) Boswellia Serrata 0.5 (10) Cetyl Dimethicone 1.5 (11) Tetrahydrocurcuminoids 0.5 (12) Shea butter 2.0 (13) Ximenia oil 1.0 (14) Zinc Peroxide-Zeolite complex 11.5 (15) Ethyl Lactate 15.0 (16) Cyclomethicone, Dimethicone Crosspolymer 2.0 (17) Polysorbate-20 2.0 (18) Polyacrylamide 2.0. Procedure. Mix (1) to (13) and heat at 70 to 80 C till homogenous. Cool to 40 to 50 C. Premix (14) to (17) and add to main batch with mixing. Cool to room temperature and add (18) and mix. An off-white cream is obtained.

### Example 8

### Skin Brightening Cleanser

Ingredients. (1) PEG-6 47.23 (2) Hydroxypropyl Guar 0.4 (3) Sodium Cocoyl Isethionate 20.0 (4) Sodium Lauryl Sulfoacetate 5.0 (5) Boswellia Serrata 0.05 (6) L-Glutathione 0.01 (7) Resveratrol 0.01 (8) Artemisinin 0.1 (9) 2,6-Dihydroxy Acetophenone 1.0 (10) Urea Peroxide-Zeolite complex 10.0 (11) Phenoxyethanol 0.7 (12) Ethylhexylglycerin 0.3 (13) Fragrance 0.2 (14) Ethylhexyl Lactate 15.0. Procedure. Mix (1) and (2) to a clear thin gel. Add (3) and (4) and mix. Premix (5) to (14). Add to main batch and mix. A white cream-like cleanser is obtained.

### Example 9

### Facial Glow Fade Cream.

Ingredients. (1) Water 72.45 (2) Dicetyl phosphate and Ceteth-10 phosphate 5.0 (3) Glyceryl Stearate and PEG-100 stearate 4.0 (4) Diglycerol 2.0 (5) Shea butter 2.0 (6) Calcium Peroxide-Zeolite complex 3.0 (7) Copper glycinate 2.2 (8) Capuacu butter 1.0 (9) Sodium hydroxide 0.25 (10) Boswellia serrata extract 0.5 (11) Tetrahydrocurcumin 0.2 (12) Paeonol 0.2 (13) Coleus Forskohlii Root extract 0.1 (14) Polysorbate-20 4.0 (15) Carnosine 0.1 (16) Preservative 1.0 (17) Polyacrylamide and C13-14 Isoparaffin and Laureth-7 2.0. Procedure. Make Premix A by mixing (1) to (5) at 80 to 90 C. Cool to 40 to 50 C and add all other ingredients and continue mixing until homogenous. Cool to room temperature.

### Example 10

### A Method of Topical Decolorizing Treatment with an Active Oxygen Donating Agent Comprising Zinc Peroxide-Zeolite Complex

The following steps are performed for this method of topical treatment. (1) The zinc peroxide 5.0 percent, water 75.0, and Sodium Potassium Aluminosilicate (Zeolite, pore size 9 Angstroms) 20.0, are mixed together. Some heat is given off at this stage and zeolite-active oxygen donating agent complex is formed. (2) The complex is applied topically in the amount necessary to achieve desired skin decolorization.

### Example 11

### A Method of Skin Whitening By Combining an Anionic Zeolite with Active Oxygen Donating Agent to Form a Complex of Zeolite with Active Oxygen Donating Agent with the Inclusion of a Solubilizing Agent

The following steps are performed. A combination of (1) PEG-6 50.0 (2) Vitamin A Palmitate 0.1 (3) Vitamin E Acetate 0.1 (4) Actiplex Botanicals 0.1 (5) Phenoxyethanol 0.5 (6) Liquapar 0.2 (7) Niacinamide 0.5, and (8) Hydroxypropyl cellulose 0.5, is mixed at 40 to 50 C for 6 hours, then (9) Zeolite (Atofina Nk30—pore size 13.0 Angstroms) 38.0 percent and zinc peroxide 10.0 percent are added and mixing continued for an additional 2 hours. The zeolite has a pore opening at least 1.0 Angstrom unit larger than the three-dimensional molecular geometry of active oxygen donating agent, which allows the entry of active agent into zeolite cavity that form a complex with zeolite.

### Example 12

### A Method for Skin Whitening Treatment via Controlled Release of Active Oxygen Donating Agent from Zeolite-Active Oxygen Donating Agent Complex

The following steps are performed. (A) A Complex is formed by mixing (1) PEG-6 45.0 (2) Dimethicone 2.0 (3) Vitamin A Palmitate 0.001 (4) Vitamin E Acetate 0.001 (5) Resacetophenone 0.01 (6) Phenoxyethanol 0.5 (7) Parabens 0.2 (8) Zinc peroxide 14.0 (9) Magnesium Aluminum Silicate 2.0 (10) Copper ATP 0.1 (11) Glutathione 0.1 (12) Licorice Root Extract 0.5, at 40 to 50 C for 6 hours, then (9) Zeolite (Atofina Nk30—pore size 9.0 Angstroms) 36.0 is added and mixing continued for an additional 2 hours. (B) The complex thus formed is applied topically. (C) Water is applied at a controlled rate as desired. Upon entry of water into said complex the complex dissociates and releases active oxygen donating agent into skin, and wherein the rate of such release is dependent on the rate of entry of water into said complex from said controlled application of water.

### BRIEF DESCRIPTION OF DRAWINGS

[FIG. 1]. The Arrangement of AlO4 and SiO4 Tetrahedra in Zeolite Cavity.

[FIG. 2]. Zeolite Cage Structures.

[FIG. 3]. Electron Photograph of Cage Complex of Zeolite with Organic Active Agent.

[FIG. 4]. Complex Formation Between A Zeolite And An Active Oxygen Donor Agent

1. A cosmetic composition comprising (i) an anionic zeolite, and (ii) an active oxygen donor agent and, (iii) wherein said zeolite forms a complex with said active oxygen donor agent and, (iv) wherein said complex releases active oxygen in a controlled-release manner, and (v) wherein said complex is for skin whitening.

2. A composition according to claim 1, wherein said zeolite is selected from a group comprising of hydrated or anhydrous aluminosilicates.

3. A composition according to claim 1, wherein said active oxygen donor agent is organic or inorganic peroxide.

4. A composition according to claim 1, wherein a carrier base is included.

5. A composition according to claim 3, wherein said organic active oxygen donor agent is selected from the group comprising urea peroxide, urea hydrogen peroxide, dibenzoyl peroxide, meta-chloroperbenzoic acid, peracetic acid, sodium peracetate, sodium meta-chloroperbenzoate, 2-butanone peroxide, di-tert-amyl peroxide, di-tert-butyl peroxide, dicumyl peroxide, dilauroyl peroxide, 2,4-Pentanedione peroxide, or combinations thereof.

6. A composition according to claim 3, wherein said inorganic active oxygen donor agent is selected from the group comprising sodium peroxide, calcium peroxide, sodium percarbonate, sodium persulfate, ammonium persulfate, barium peroxide, magnesium peroxide, lithium peroxide, zinc peroxide, or combinations thereof.

7. A composition according to claim 3, wherein said organic active oxygen donor agent is Dibenzoyl peroxide.

8. A composition according to claim 3, wherein said inorganic active oxygen donor agent is zinc peroxide.

9. A composition according to claim 3, wherein said inorganic active oxygen donor agent is sodium percarbonate.

10. A composition according to claim 4, wherein a carrier base is a cleanser composition.

11. A method of cosmetic treatment with a zeolite-active oxygen donor agent complex, and comprising; (i) the contact of at least one anionic zeolite and at least one active oxygen donor agent to form a complex of said zeolite and said active oxygen donor agent, and (ii) the topical application of said complex, and (iii) repeating of steps (i) to (ii) until desired treatment is obtained.

12. A composition according to claim 11, wherein said zeolite is selected from a group comprising of aluminosilicates that is either in hydrated or in anhydrous form.

13. A composition according to claim 11, wherein said active oxygen donor agent is organic or inorganic peroxide.

14. A composition according to claim 11, wherein said treatment is for skin whitening.

15. A composition according to claim 13, wherein said organic active oxygen donor agent is selected from the group comprising urea peroxide, urea hydrogen peroxide, dibenzoyl peroxide, meta-chloroperbenzoic acid, peracetic acid, sodium peracetate, sodium meta-chloroperbenzoate, 2-butanone peroxide, di-tert-amyl peroxide, di-tert-butyl peroxide, dicumyl peroxide, dilauroyl peroxide, 2,4-Pentanedione peroxide, or combinations thereof.

16. A composition according to claim 13, wherein said inorganic active oxygen donor agent is selected from the group comprising sodium peroxide, calcium peroxide, sodium percarbonate, sodium persulfate, ammonium persulfate, barium peroxide, magnesium peroxide, lithium peroxide, zinc peroxide, or combinations thereof.

17. A composition according to claim 13, wherein said organic active oxygen donor agent is Dibenzoyl peroxide.

18. A composition according to claim 13, wherein said inorganic active oxygen donor agent is zinc peroxide.

19. A composition according to claim 13, wherein said inorganic active oxygen donor agent is sodium percarbonate.

20. A composition according to claim 11, wherein said treatment is for reduction of topical discoloration including sun-spot and age-spot.
